(19) United States
(12) Patent Application Publication
(10) Pub. No.: US 2025/0302392 A1
(43) Pub. Date: Oct. 2, 2025

(54) Pulse Waveform-Based Detection and Categorization of Cardiovascular Anomalies

(71) Applicant: LifeQ B.V., Amsterdam (NL)

(72) Inventors: Laurence Richard Olivier, Alpharetta, GA (US); Franco Du Preez, Cumming, GA (US); Jacobus Barend Van Dyk, Malmesbury (ZA)

(21) Appl. No.: 18/864,170

(22) PCT Filed: May 11, 2023
(86) PCT No.: PCT/US2023/066870

§ 371 Date: Nov. 8, 2024

Related U.S. Application Data
(60) Provisional application No. 63/364,520, filed on May 11, 2022.

(51) Int. Cl. A61B 5/00; A61B 5/02; A61B 5/1455; G16H 50/20; G16H 50/30; G16H 50/70

(52) U.S. Cl. CPC A61B 5/7267; A61B 5/02007; A61B 5/02028; A61B 5/14552; A61B 5/6816; A61B 5/6824; A61B 5/6826; A61B 5/7239; A61B 5/7257; A61B 5/742; G16H 50/20; G16H 50/30; G16H 50/70; A61B 2560/0462

(57) ABSTRACT

A method for constructing and implementing an anomaly detection system for the evaluation of the health status of users through the analysis of their PPG signal measured using wearable technology. In one embodiment of this anomaly detection system, a convolutional neural network deep learning model is used to derive a feature vector of the PPG signal and construct a low dimensional feature map along with known cardiovascular health concerns to identify possible health concerns in users of unknown health status.

### BACKGROUND

Since the 1870's the value of the pulse waveform in medical applications has become apparent. F. A. Mahomed stated in 1872 that the information contained in the pulse waveform is of such importance and is so regularly consulted that it must be to the advantage of medical professionals to fully appreciate the pulse waveform, and to extract from it as much detail as is possible. Since then, the pulse waveform has become a regular health monitoring datastream in the medical profession and is used for the monitoring of vital signs such as, but not limited to, heart rate, cardiac cycle, respiration, the depth of anesthesia, and blood pressure. Analysis of the pulse waveform has also been used for the development of monitoring applications in the broader health, wellness and medicine (HWM) industry. This allows for the continuous monitoring of several medically relevant vitals, such as, but not limited to, heart rate, breathing rate, and oxygen saturation (SpO₂).

### SUMMARY OF INVENTION

Embodiments of the claimed invention comprise a method for the evaluation of a user's cardiovascular health status using anomaly detection techniques to interpret photoplethysmography (PPG) signal data obtained through wearable devices, in conjunction with more conventional methods of analyzing PPG signal data to provide feedback to users and interested third parties such as medical practitioners by making the resulting information available to them. The PPG signal obtained from the wearable devices requires some digital signal processing to filter, detrend and de-noise the data prior to analysis. In one aspect, the PPG signal may be divided into segments of equal length prior to being fed into the anomaly detection system for training or analyzing the PPG signal data using a fully trained anomaly detection system. In some instances, the conventional analysis of the PPG signal may be used to improve the feedback given to the user or interested third party. Embodiments of the claimed invention may aid medical practitioners to remotely monitor patients diagnosed with cardiovascular health concerns, through continuous collection and analysis of PPG signal data using wearable devices, and to evaluate the success of recommended medical interventions.

According to one aspect, the invention is directed at a method for identifying a health status of a user using an IoT setup of interconnected electronic devices and sensors. The method can include collecting a user's PPG signal using a wearable device, preprocessing the PPG signal for a conventional analysis of the PPG signal and extraction of critical points and interpretable engineered one dimensional (1D) features such as, but not limited to, crest time, SEVR, ejection duration index, large artery stiffness index, small artery resistance and features relating to hypertension. After the preprocessing, the method can utilize an anomaly detection system, which can be exemplified by a convolutional neural network trained on pulse waveform data, to produce a feature vector in a latent space. A dimension reduction method can then be used to construct a low dimensional representation (two-dimensional or three-dimensional) of the feature vector. 21. The dimension reduction method can include comprises PCA or t-SNE.

Sections of the two-dimensional/three-dimensional space can then be labeled as corresponding to healthy, specific condition or unknown based on the class assigned to the PPG signal by the anomaly detection system. Then interpretable engineered 1D features can be created that refer to specific physiological processes associated with health risk. These interpretable engineered features can be used together with the healthy/disease/unknown output of the anomaly detection system, to resolve ‘unknown’ anomalies as being healthy or unhealthy based on whether the interpretable engineered features have 1D values associated either health or specific condition. Further, feedback can be provided to the user, or to a third party, regarding the health states of the user where in the case of an unknown label output.

According to an aspect, conventional analysis of the PPG signal may be used to determine the critical points associated with the PPG pulse waveform and its derivatives, calculate the exhaustive set of features from the critical points, determine the health related and interpretable engineered one dimensional (1D) features from the exhaustive set of features calculated from the critical points, and derive a small subset of interpretable features related to independent aspects of health and anatomy. The critical point associated with the PPG, VPG and APG signals can be determined using the two-moving-average method. Further, an exhaustive set of features can be derived from the difference between any two critical points in the form of amplitude, timespan, subarea and slope features. In some aspects, the interpretable engineered one dimensional (1D) features comprise crest time, normalized crest time, SEVR, ejection duration, ejection duration index, large artery stiffness index, small artery resistance and features relating to hypertension are derived from the exhaustive set of features calculated from the x- and y-coordinates of the critical points. Techniques for setting up covariance matrices, or principal component analysis are used to remove interpretable engineered one-dimensional (1D) features with covariance, or to select interpretable engineered one dimensional (1D) features that holds unique health or anatomy related information.

In an aspect, a convolutional neural network can be constructed from a one dimensional or two-dimensional representation (via frequency domain methods, e.g. Fourier spectrum) of the PPG signal, for the extraction of feature sets from the PPG signal, for the classification of these feature sets into health state classes, and for placement of the health states in the low dimensional representative manifold. The input data derived from segments of the PPG signal can be preprocessed and converted into a one-dimensional representation of the PPG signal, or are used to derive a two dimensional frequency domain representation of the PPG including Fourier spectrum, Lomb-Scargle periodogram or by cardiopulmonary coupling. Further, a one- or two-dimensional representation of the PPG signal data can be used as input data to train the CNN and to obtain the output feature set which is mapped onto the low dimensional representative framework using methods including t-SNE or PCA. A loss function can be used to organize the manifold and to create continuity in the feature set classes, by identifying the health state regions in the low dimensional representative manifold, wherein the loss function includes triplet loss, mean square error loss, or cross entropy loss.

In an aspect, a user's CNN feature set corresponding to an unknown region in the low dimensional representative manifold can be compared to the interpretable engineered one dimensional (1D) features containing unique health and anatomy information as derived through the conventional analysis of the PPG signal, to evaluate the health state of the user. The unknown regions in the low dimensional representative manifold can be assigned based on the interpretable engineered one dimensional (1D) features containing unique health and anatomy information as derived through the conventional analysis of the PPG signal corresponding to those unknown regions.

Feedback can be given to users and interested third parties using displays including desktop computer display, laptop display, smartphone display, wearable device display, phone calls, text messages, emails, or web-based dashboards. the computational aspects of the invention can be performed remotely on devices such as, but not limited to, smart wearable device, smartphone, desktop computer, laptop, or may be performed using cloud computing infrastructure. The wearable device can include a fingertip pulse oximeter, an earlobe pulse oximeter, a wrist worn wearable device, or a smart ring. 21. The method of claim 1, wherein the dimension reduction method comprises PCA or t-SNE.?

According to another aspect of the present disclosure, the method is directed at a method for identifying a health status of a user using an IoT setup of interconnected electronic devices and sensors. The method includes collecting a user's PPG signal using a wearable device and preprocessing the PPG signal for a conventional analysis of the PPG signal to extract at least one interpretable engineered one dimensional (1D) feature from the PPG signal. An anomaly detection system can be used on the PPG signal to produce a feature vector in a latent space and to produce a classification of the PPG signal corresponding to a healthy state or a state associated with another condition. A low dimensional representation can then be created of the feature vector by applying PCA or t-SNE to the feature vector. The low dimensional representation of the PPG signal can be labeled to correspond to the state classified by the anomaly detection system. Then, the classifications can be associated with the interpretable engineered 1D features in the low dimensional representation space. Next, anomalies labeled as unknown can be resolved as belonging to a healthy state or an associated with another condition. Then, feedback can be provided to the user, or to a third party, regarding a health state of the user. In an aspect, the wearable device comprises one of a fingertip pulse oximeter, an earlobe pulse oximeter, a wrist worn wearable device, or a smart ring.

In some aspects, interpretable engineered 1D features can include crest time, SEVR, ejection duration index, large artery stiffness index, small artery resistance and features relating to hypertension. In addition, the anomaly detection system includes a convolutional neural network trained on PPG signal data from other users.

In an aspect, the invention is directed towards identifying a health status of a user using an IoT setup of interconnected electronic devices and sensors. The method can include collecting a user's PPG signal using at least one of wearable devices. The wearable devices can include, but are limited to, fingertip pulse oximeters, earlobe pulse oximeter, wrist worn wearable devices, or smart rings.

The next step can include preprocessing the PPG signal for the conventional analysis of the PPG signal. The preprocessing can include the application of preprocessing filters such as, but not limited to an inverse Chebyshev filter, or a Butterworths filter to improve signal quality. In addition, VPG and APG can be derived from the PPG signal. Also, a two-moving averages method can be used for the extraction of the critical points associated with the PPG, VPG and APG signals. In an aspect, conventional analysis of the PPG signal may be used to determine the critical points associated with the PPG pulse waveform and its derivatives, calculate the exhaustive set of features from the critical points, determine the health related and interpretable engineered one dimensional (1D) features from the exhaustive set of features calculated from the critical points, and derive a small subset of interpretable features related to independent aspects of health and anatomy.

After preprocessing, determining interpretable engineered one dimensional (1D) features such as, but not limited to crest time, SEVR, ejection duration index, large artery stiffness index, small artery resistance, and features relating to hypertension can be done. From here, an anomaly detection system can be used on the PPG signal to produce a feature vector in a latent space and to produce a classification of the PPG signal corresponding to a healthy state or a state associated with another condition. A low dimensional representation of the feature vector can then be constructed, with the labeling the low dimensional representation of the PPG signal corresponding to the state classified by the anomaly detection system to follow. Then, associating the interpretable engineered 1D features with the classification in the low dimensional representation space. Next, anomalies labeled as unknown can be resolved as belonging to a healthy state or an associated with another condition. Feedback can then be provided, to either the user, or to a third party, regarding a health state of the user.

In an aspect, the interpretable engineered one dimensional (1D) features can include crest time, normalized crest time, SEVR, ejection duration, ejection duration index, large artery stiffness index, small artery resistance and features relating to hypertension are derived from the exhaustive set of features calculated from the x- and y-coordinates of the critical points. Further, techniques for setting up covariance matrices, or principal component analysis can be used to remove interpretable engineered one-dimensional (1D) features with covariance, or to select interpretable engineered one dimensional (1D) features that holds unique health or anatomy related information.

In some aspects, a convolutional neural network can be constructed from a one dimensional or two-dimensional representation (via frequency domain methods, e.g. Fourier spectrum) of the PPG signal. The convolutional neural network can be used for the extraction of feature sets from the PPG signal, for the classification of these feature sets into health state classes, and/or for placement of the health states in the low dimensional representative manifold. Further, the input data derived from segments of the PPG signal can be preprocessed and converted into a one-dimensional representation of the PPG signal, or are used to derive a two dimensional frequency domain representation of the PPG including Fourier spectrum, Lomb-Scargle periodogram or by cardiopulmonary coupling. In some aspects, a one- or two-dimensional representation of the PPG signal data is used as input data to train the CNN and to obtain the output feature set which is mapped onto the low dimensional representative framework using methods including t-SNE or PCA. In addition, a loss function can be used to organize the manifold and to create continuity in the feature set classes, by identifying the health state regions in the low dimensional representative manifold, wherein the loss function includes triplet loss, mean square error loss, or cross entropy loss.

In some aspects, a user's CNN feature set corresponding to an unknown region in the low dimensional representative manifold can be compared to the interpretable engineered one dimensional (1D) features small subset of features containing unique health and anatomy information as derived through the conventional analysis of the PPG signal, to evaluate the health state of the user.

In an aspect, the disclosure is directed at an IoT system of interconnected devices and sensors. The system collects a user's PPG signal with a wearable device. The PPG signals are preprocessed to extract critical points and interpretable 1D features such as crest time, SEVR, ejection duration index, large artery stiffness index, and small artery resistance. An anomaly detection system is used to produce a feature vector from the CNN trained on pulse waveform data. The system can then construct a 2D or 3D representation of the feature vector through dimension reduction, and then can label the 2D/3D space as healthy, specific condition, or unknown based on the class assigned by the anomaly detection system. From here, the system can then create interpretable 1D features related to physiological processes associated with health risk and then assign health status by combining the output of the anomaly detection system and the interpretable 1D features. The system can then provide feedback to the user or a third party on the health status.

In an aspect, the conventional analysis of the PPG signal can include determining critical points in the PPG signal and its derivatives, calculating an exhaustive set of features from the critical points, extracting interpretable 1D features from the exhaustive set, and selecting a subset of interpretable features related to health and anatomy. The critical points can be determined using the two-moving-average method. Features can be derived from the difference between any two critical points, including amplitude, timespan, subarea, and slope features. The interpretable 1D features can include crest time, SEVR, ejection duration index, large artery stiffness index, and small artery resistance.

In such aspects, the system can construct a CNN to extract feature sets from the PPG signal, classify the feature sets into health state classes, and map the health states to a low dimensional representative space. From here, the CNN can be trained on a 1D or 2D representation of the PPG signal, and the output feature set is mapped to a low dimensional representative space using methods such as t-SNE or PCA. In some aspects, a loss function is used to organize the low dimensional representative space and identify health state regions, including triplet loss, mean square error loss, or cross entropy loss. Unknown CNN feature sets can be compared to the interpretable 1D features to evaluate the user's health status.

It is to be understood that this summary is not an extensive overview of the disclosure. This summary is exemplary and not restrictive, and it is intended to neither identify key or critical elements of the disclosure nor delineate the scope thereof. The sole purpose of this summary is to explain and exemplify certain concepts of the disclosure as an introduction to the following complete and extensive detailed description.

### BRIEF DESCRIPTION OF DRAWINGS

The features and components of the following figures are illustrated to emphasize the general principles of the present disclosure. Corresponding features and components throughout the figures can be designated by matching reference characters for the sake of consistency and clarity.

FIG. 1 is a schematic representation of an embodiment of the invention.

FIG. 2 is an illustration of the PPG, VPG and APG signals and the location of the critical points.

FIG. 3 is an exemplary method for the determination of a subset of interpretable features related to independent aspects of health and anatomy.

FIG. 4 is an illustration of how the anomaly detection system may be implemented using a manifold in the CNN's embedding space.

FIG. 5 is a schematic representation of the IoT setup for the operation of the invention.

FIG. 6 is an illustration of how the two-moving-average method may be applied to the PPG pulse waveform.

### DETAILED DESCRIPTION

### Definitions

CNN—Convolutional neural network
IoT—Internet of all things
SIANN—Shift/Space invariant artificial neural network
PPG—photoplethysmography
VPG—velocity plethysmography
APG—acceleration plethysmography
AWS—Amazon web services
t-SNE—t-distributed stochastic neighbor embedding
PCA—principal component analysis
Afib—Atrial fibrillation
GAN—Generative adversarial network
ResNeXt—50 layers deep neural network model developed for the identification of atrial fibrillation using PPG signal data.
De-noising—the process of removing unwanted modifications, or noise, from the signal data arising during the measurement of signal data.

It should be appreciated that this disclosure is not limited to the systems and components described herein. It is also to be understood that the terminology used herein is for the purpose of describing certain embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Any systems and components similar or equivalent to those described herein can be used in the practice or testing of the present invention. All publications mentioned are incorporated herein by reference in their entirety.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

The pulse waveform can be measured by technologies such as photoplethysmography (PPG), employed by various wearable and non-wearable pulse oximeter devices, wearable earlobe pulse oximeter devices, as well as in smart wearable devices such as wrist worn devices and smart rings. The PPG signal is obtained by illuminating the skin and measuring the changes in light absorption brought about by the perfusion of blood to the dermis and subcutaneous tissue of the skin. Blood is pumped to the dermis and subcutaneous tissue of the skin with each cardiac cycle resulting in a pressure pulse that moves through the arteries and arterioles. The pressure pulse causes a change in volume in the arteries and arterioles of the subcutaneous tissue which can be detected by illuminating the skin with the light from a light-emitting diode (LED). The amount of light either transmitted, as is the case in fingertip pulse oximeters, or reflected, as in the case in wrist worn wearable devices, to a photodiode can then be measured. The resulting PPG signal appears as a series of peaks with each peak resulting from a cardiac cycle.

The PPG signal obtained from wearable devices reflects the movement of blood in the blood vessels of the subcutaneous tissue, which moves from the heart to the dermis, and subcutaneous tissue of the skin, where the wearable is placed. The wave-like motion of the blood flow pressure pulse alters the amount of light transmitted through the extremity where the wearable is placed or alters the amount of backscattering of light to the photodiode of the wearable. This alteration on light reaching the photodiode corresponds with the variation of the blood volume in the pressure pulse.

The PPG signal captures the wave-like motion of the blood flow pressure pulse continuously, which gives rise to a pressure pulse corresponding to each heartbeat. Each heartbeat corresponds to a pulse waveform that captures characteristics of the heart during the corresponding heartbeat. The resulting arterial pulse waveform is composed of three distinct components which display heart function: (1) systolic phase; (2) dicrotic phase; and (3) diastolic phase.

The systolic phase of the pulse waveform is characterized by a rapid increase in the pressure and increases until it reaches a maximum pressure, referred to as the systolic peak(S), followed by a decrease in the pressure pulse. The systolic phase is initiated by the opening of the aortic valve and corresponds to the left ventricular ejection. The next component of the pulse waveform is referred to as the dicrotic notch (N) and is widely believed to correspond to the closure of the aortic valve. The third component of the pulse waveform is referred to as the diastolic phase. The diastolic phase represents the run-off of blood into the peripheral circulatory system and is characterized by a secondary peak with the maximum pressure reached in the diastolic phase corresponding to the diastolic peak (D). The shape of the pulse waveform is affected by multiple factors, such as the hemodynamics and the physiological conditions caused by the change in the properties of the arterioles.

The critical points are a selection of points of interest in the pulse waveform corresponding to maximum and minimum points, or the start and end points, of the pulse waveform that may contain valuable physiologically relevant information regarding the functioning of the heart. The critical points of onset of the pulse waveform (O), the maximum associated with the systolic peak(S), the minimum associated with the dicrotic notch (N), the maximum associated with the diastolic peak (D), and the endpoint of the pulse waveform (E) corresponding to the O point of the following pulse waveform, can be determined using methods such as that laid out by Dr. Elgendi. See Elgendi M. TERMA framework for biomedical signal analysis: An economic-inspired approach. J. Biosensors, 2016, 6 (4): 55. These methods are used for the conventional analysis of the Pulse Waveforms to derive physiologically relevant features to compare to the low dimensional representation of the CNN analysis.

Further critical points may be identified by taking the first derivative of the PPG signal referred to as the velocity plethysmograph (VPG). The critical points of the maximum positive velocity in the systolic phase (w), the minimum negative velocity in the systolic phase (y), and the maximum positive velocity in the diastolic phase (z) can be determined from the VPG signal. Similarly, the second derivative of the PPG signal referred to as the acceleration plethysmograph (APG) may be used to derive the critical points associated with the a to e waves, referred to here as a, b, c, d, and e. Identification of the critical points associated with the pulse waveform relies on the application of biomedical analytical techniques.

In a publication by Dr. Elgendi. Elgendi M. TERMA framework for biomedical signal analysis: An economic-inspired approach. J. Biosensors, 2016, 6 (4): 55. a framework for the analysis of the PPG biomedical signal was laid out. Even though the analysis of biomedical signals, including PPG signals, have been developed over the past 20 years, this publication aimed to develop a standardized framework for the analysis of biomedical signals. Methods such as that laid out by Dr. Elgendi may be used for the determination of the critical points associated with the pulse waveform.

The exemplary embodiments described herein are provided for illustrative purposes and are not limiting. Other exemplary embodiments are possible, and modifications may be made to the exemplary embodiments within the spirit and scope of the disclosure. Therefore, the Detailed Description is not meant to limit the disclosure. Rather the scope of the disclosure is defined only in accordance with the following claims and their equivalents. The following detailed description of the exemplary embodiments will so fully reveal the general nature of the disclosure that others can, by applying knowledge of those skilled in the relevant art(s), readily modify and/or adapt for various applications such exemplary embodiments, without undue experimentation, without departing from the spirit and scope of the disclosure. Therefore, such adaptations and modifications are intended to be within the meaning and plurality of equivalents of the exemplary embodiments based upon the teaching and guidance presented herein. It is to be understood that the phraseology or terminology herein is for the purpose of description and not of limitation, such that the terminology or phraseology of the present specification is to be interpreted by those skilled in relevant art(s) in light of the teachings herein.

In some instances, analysis of the pulse waveform and identification of the critical points associated with the pulse waveform may require the addition of preprocessing filters such as, but not limited to, the inverse Chebyshev filter, or the Butterworth filter, to improve signal quality and aid in the identification of specific critical points, such as the dicrotic notch (N), which may be hard to detect in some pulse waveforms.

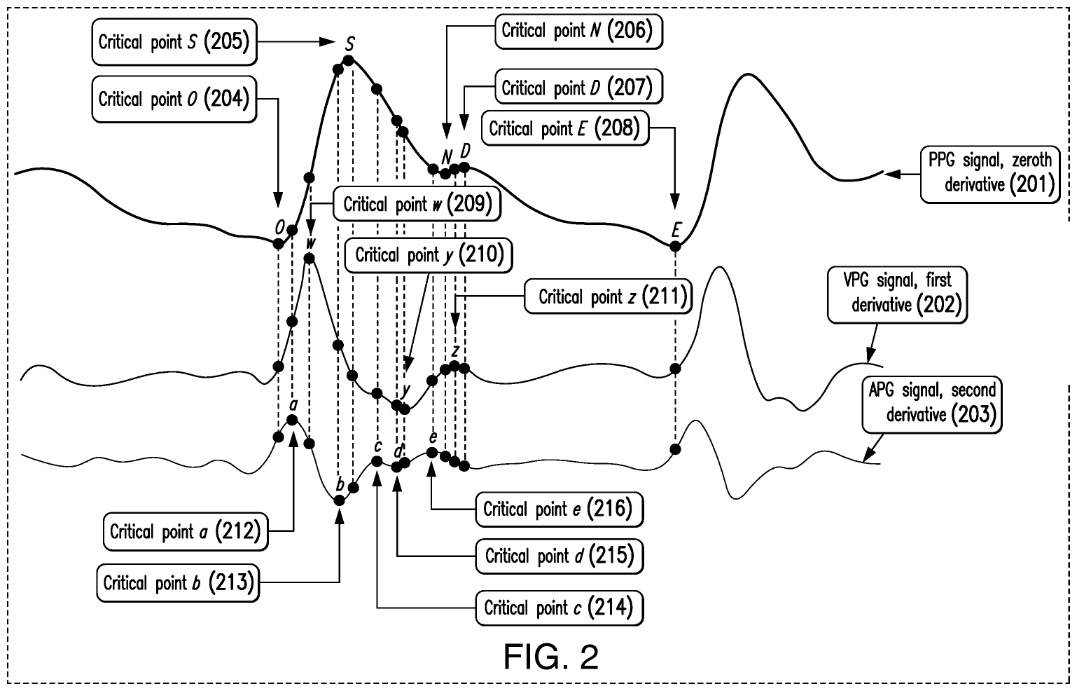

In an aspect, the identification of the critical points for the pulse waveform yields a set of 13 critical points derived from the PPG, VPG and APG signals with an x-coordinate and y-coordinate for each critical point as illustrated in FIG. 2. The methods used for the identification of the critical points associated with the pulse waveform may be performed on every pulse waveform in a series of pulse waveforms obtained for a user to derive the x and y coordinates of each critical point for each pulse waveform in a series of pulse waveforms. This entails the isolation of each pulse waveform in the series and zeroing of each isolated pulse waveform. Alternatively, a series of pulse waveforms may be used to derive a representative pulse waveform, for instance a 30 second series of pulse waveforms may be used and aggregated to obtain a single representative pulse waveform for each 30 second series.

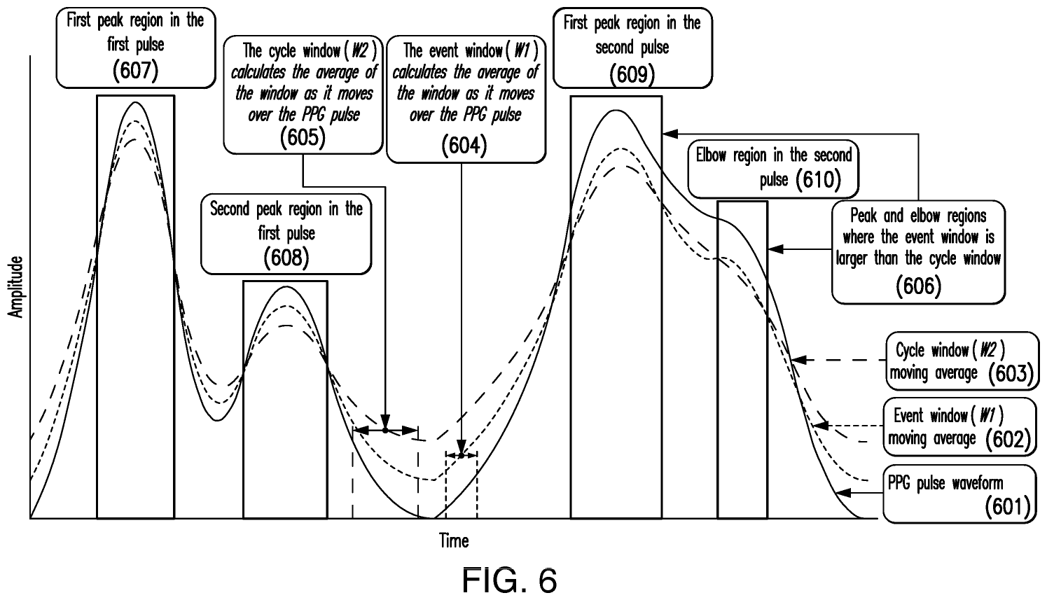

Methods such as, but not limited to, the two-moving-average method as described by Dr. Elgendi may be used to determine the position of the critical points in the PPG, VPG and APG signals of the isolated pulse waveforms respectively. This is illustrated in FIG. 6 for the analysis of the PPG pulse waveform (601). The two-moving-average method entails using two aggregation windows of different sizes to calculate the moving average of the aggregation window over the pulse waveform. The smaller aggregation window, referred to here as W₁, is the event window which captures the peak and elbow of the pulse corresponding to the moving average result calculate over the event window width (602), whereas the larger aggregation window, referred to here as W₂, is the cycle window which emphasizes the region that contains the peaks and elbows, the moving average is calculated over the cycle window width (603). The smooth convolution operation is applied to the middle point in the moving aggregation windows for both W₁ (604) and W₂ (605). To further illustrate how this method may be applied, consider a certain point in the beginning of the pulse waveform, the moving-average windows generate two different mean values due to the windows (W₁, W₂) including different regions of the pulse waveform. Since the cycle window W₂, which is the larger of the two windows, considers part of the systolic peak region, the convolution of W₂ will be larger than the convolution of the event window W₁. As the two windows slide to the systolic peak region, W₂ will include smaller edge points from the start of the PPG pulse waveform, in comparison to W₁, resulting in a smaller convolution than the convolution of W₁, as can be seen in the first block region in the first PPG pulse in FIG. 6 (607). This two-moving-average method yields block regions where the convolution of W₁ is larger than the convolution of W₂, which can be considered as peak or elbow regions (606). FIG. 6 illustrates the application of the two-moving average method to two PPG pulses resulting in four peak and elbow regions. The first PPG pulse indicates a first and second peak region (607 and 608), whereas the second pulse indicates a peak region (609) corresponding with the systolic peak and an elbow region (610) corresponding to the diastolic peak. The critical points are contained in the peak and elbow regions of the PPG, VPG, and APG.

The ability to obtain the x- and y-coordinates for each critical point for each pulse waveform, or representative pulse waveform, in a series of pulse waveforms allows for the derivation of further features to describe the PPG signal. In an exemplary embodiment of this invention, a series of amplitude features may be derived by recording the difference between the y-coordinates of any and all two critical points of the PPG, VPG, and APG, yielding a total of 211 amplitude features, as discussed below.

As shown in FIG. 2, the critical points detected in one curve can be marked in the other two curves at the same moment. For example, the w (209) point can be marked in the PPG and the difference between the y-coordinates of the w (209) point and S (205) point can be calculated as an amplitude feature. In the PPG pulse waveform, any two critical points with the exception of O (204) and E (208) points (the amplitude in O and E points are normalized to 0 in PPG) can generate an amplitude ratio feature. There are 55 (11×10/2=55) amplitude features exploited in the PPG pulse waveform by combination calculations from 11 critical points. Similarly, in the VPG or APG, any two critical points can generate an amplitude ratio feature. Since the y-coordinates of the O (204) and E (208) points are not 0 in VPG (202) and APG (203), there are 78 (13×12/2-78, combination calculation from 13 critical points) amplitude features exploited in VPG or APG pulse waveforms. In total, we have 211 amplitude features. Furthermore, a series of timespan features may be derived by recording the difference between the x-coordinates of any and all two critical points, yielding a total of 77 timespan features (13×12/2−1=77, excluding timespan between O (204) and E (208) point.).

The pulse waveform can also be divided into subsections between the different critical points where any two critical points can generate an area under the pulse waveform subsection. Each sub-area may or may not be normalized by the total area under the entire pulse waveform and each sub-area may be integrated using methods such as, but not limited to, numerical integration, or integration methods based on the trapezoidal rule. By combination calculation from 13 critical points (13×12/2−1=77, excluding total area between O (204) and E (208) points.), the sub-area features derived by integration of the sub-areas of the pulse waveform yields a total of 77 sub-area features. Another set of features that may or may not be derived from the set of critical points is the slope between any two critical points, which reflects the rate of the shape change in the pulse waveform between the specified two critical points. Similar to the previous combination calculation, this calculation yields a total of 77 (13×12/2−1=77, excluding total area between O (204) and E (208) points) slope features derived from the pulse waveform.

Methods for the determination of amplitude features, timespan features, sub-area features, and slope features may be employed to derive a total of 445 features that give a detailed description of the pulse waveform. These features may be used to derive an exhaustive set of combinations and ratios of critical point features. In one aspect, this exhaustive set of critical point features may be used in machine learning applications such as, but not limited to, disease state monitoring (including cardiovascular focused MLA), and general anomaly detection in the HWM industry, or other health related applications which may become available in the future. Furthermore, select features in this exhaustive set of critical point features may have been shown to strongly correlate with known health conditions or diseased states and these features may be given to the users or interested third parties for the evaluation of a user's health. Some of these features will be discussed here as it is relevant to the current invention.

The systolic phase of the PPG pulse waveform represents the cardiac output which is the product of heart rate and stroke volume from the heart. The stroke volume is determined by the left ventricular filling and left ventricular function. The crest time, which is defined as the time from the foot of the PPG pulse waveform (O) to the systolic peak(S) reflects how fast the left ventricular filling is and how well the left ventricular function performs. The stronger and more elastic the cardiac muscle is, the faster the left ventricle can inject stroke volume into the aorta and therefore the shorter the crest time is and the healthier the subject is. The crest time may be positively influenced by youth and intense exercise which corresponds to better myocardium function in a user that is young and fit as compared to a user that is old and unfit. Furthermore, several cardiovascular diseases such as, but not limited to, aortic valve stenosis and regurgitation, and mitral valve disorder may have an influence on crest time. The crest time may be of interest to users, or interested third parties since it is, in general terms, an indication of myocardial function. It has been established that the normalized crest time as derived from the PPG signal of healthy individuals presents a relative average value of below 0.2, whereas individuals with acute myocardial infarction (AMI), chronic myocardial infarction (CMI) and antiphospholipid syndrome (SAA) have a higher mean normalized crest time value. See e.g., Angius, Gianmarco, Doris Barcellona, Elisa Cauli, Luigi Meloni, and Luigi Raffo. “Myocardial infarction and antiphospholipid syndrome: a first study on finger PPG waveforms effects.” In 2012 Computing in Cardiology, pp. 517-520. IEEE, 2012. The crest time can be expressed as the absolute crest time as calculated using Equation 1, or the normalized crest time as calculated using Equation 2.

$$Absolute crest time = ( {S}_{x} - {O}_{x} ) \qquad [ Equation 1 ]$$

$$Normalized crest time = ( {S}_{x} - {O}_{x} ) / ( {E}_{x} - {O}_{x} ) \qquad [ Equation 2 ]$$

Another feature of importance to users and interested third parties in the HWM industry is the subendocardial viability ratio (SEVR) which is calculated as the estimated ratio of myocardial perfusion relative to cardiac workload and is calculated as the ratio of diastolic pressure-time index (DPTI) over the systolic pressure-time index (SPTI). The physiological meaning of SEVR relies on a background knowledge of cardiac circulation. The systemic circulation is composed of one engine and two pumps. The first pump is the left ventricle, which represents the systolic pump. The second one is the aorta and large elastic arteries, which represent the diastolic pump. In the systolic phase, the left ventricle acts as a pump to push the blood stroke into the aorta and the expanded aorta stores part of the stroke. Subsequently, the large elastic aorta acts as another pump to push the stored stroke into the other vessels in the diastolic phase. The coronary artery of the heart cannot be perfused during the contraction phase (systolic phase) due to the extravascular compressive forces in the cardiac muscle. So subendocardial perfusion occurs only during the diastolic phase of the cardiac cycle. The low pressure from the aorta brings the opportunity to pump blood into the coronary artery to support the heart function. The area between the aortic and left ventricular pressure curves in the diastole represents the pressure that affects the coronary blood flow and maintains adequate subendocardial blood supply in the diastolic phase of the cardiac cycle. If the DPTI is small, it means a reduction in diastolic blood pressure in the aorta and thus a reduction in subendocardial perfusion. The less blood supply into the coronary arteries, the more cardiac afterload increases, resulting in heart overload.

The area under the left ventricular pressure waveform in systole, from the onset of the ventricular systole to the dicrotic notch, represents the left ventricular afterload and defines the cardiac workload. In the case where the mean arterial pressure during the systolic phase in the ascending aorta is high, the left ventricle must contract more energetically to maintain adequate stroke volume. Therefore, the systolic area describes the myocardial oxygen requirements and depends predominantly on the left ventricular ejection time, ejection pressure and the myocardial contractility. The area between the aortic and left ventricular pressure curves in the diastole represents the pressure that affects the coronary blood flow and maintains adequate subendocardial blood supply in the diastolic phase of the cardiac cycle. This indicates the degree of heart perfusion: the heart cannot be perfused during contraction due to the high pressure, but the diastolic cycle with low pressure brings the opportunity to pump blood into the coronary artery that feeds out from the base of the Aorta. The SEVR may be calculated from the subarea features obtained from the PPG pulse waveform. The ratio between the subarea features ON and NE in the PPG pulse waveform is used to calculate SEVR, as is shown in Equation 3.

$$SEVR = DPT 1 / SPTI = subarea ( NE ) / subarea ( ON ) \qquad [ Equation 3 ]$$

The third feature indicating heart muscle health is ejection duration index which is calculated as the normalized timespan from the foot (O) of the PPG pulse waveform to the dicrotic notch time (N). The left ventricular ejection duration is the time elapsing from the start of the left ventricular contraction till closure of the aortic valve and is the phase of systole duration. The ejection duration has been used to assess left ventricular function and contractility. It not only indicates the strength of heart muscle similar to crest time, but also reflects the contraction and blood ejection functions of the left ventricular chamber. Heart ventricular failure may result from both a very short ejection duration and long ejection duration. If the left ventricular chamber is abnormally enlarged, the left ventricular chamber muscle becomes thinner and weaker, resulting in more blood to be filled and a reduction in the constriction speed. Therefore, the systole phase will increase and result in prolonged ejection duration. This is called systolic dysfunction.

In contrast, under diastolic dysfunction, the chamber muscle is more stiff and thicker, and the chamber volume is decreased, resulting in less blood being ejected into the aorta. In this case the ejection duration will be shorter in comparison to the normal case. Furthermore, several other cardiovascular diseases may cause an increase in the ejection duration, such as aortic valve stenosis (Pagoulatou, Stamatia, Nikolaos Stergiopulos, Vasiliki Bikia, Georgios Rovas, Marc-Joseph Licker, Hajo Müller, Stéphane Noble, and Dionysios Adamopoulos. “Acute effects of transcatheter aortic valve replacement on the ventricular-aortic interaction.” American Journal of Physiology-Heart and Circulatory Physiology 319, no. 6 (2020): H1451-H1458.), aortic valve regurgitation (Kamran, Haroon, Louis Salciccioli, Carl-Frederic Bastien, Abhishek Sharma, and Jason M. Lazar. “The association between aortic regurgitation and increased arterial wave reflection.” Artery Research 6, no. 1 (2012): 49-54.), and ascending aortic aneurysm (Salvi, Lucia, Jacopo Alfonsi, Andrea Grillo, Alessandro Pini, Davide Soranna, Antonella Zambon, Davide Pacini, Roberto Di Bartolomeo, Paolo Salvi, and Gianfranco Parati. “Postoperative and mid-term hemodynamic changes after replacement of the ascending aorta.” The Journal of Thoracic and Cardiovascular Surgery (2020)). The ejection duration and ejection duration index may be calculated using Equations 4 and 5, respectively.

$$Ejection duration = ( {N}_{x} - {O}_{x} ) \qquad [ Equation 4 ]$$

$$Ejection duration index = ( {N}_{x} - {O}_{x} ) / ( {E}_{x} - {O}_{x} ) \qquad [ Equation 5 ]$$

The stiffness index, which is defined as the height of the subject divided by the time difference between the systolic peak and the diastolic peak, where the time difference is calculated as the timespan between the critical points S and D divided by the sampling rate, described by Equation 6. As described above, the shape of the pulse waveform is determined by the left ventricle and the aorta. However, the relationship between the left ventricle and the aorta cannot explain all the phenomena defining blood pressure and pulse waveform and the wave reflection also contributes to the shape of the PPG pulse waveform detected at the extremities of the body. Considering the physiological relevance of the stiffness index it may be of convenience to relate this phenomenon with a basin full of water, and a series of concentric waves traveling from the center point to the edges of the basin. The first wave will move back towards the center of the basin after hitting the external edges. This backward wave will superimpose on the second centrifugal wave, generating much larger waves. In a similar fashion the forward wave generated by the heart pump travels along the different pipelines (the aorta, arteries, arterioles, capillaries, etc.).

Some typical reflection sites include arterial bifurcations, atherosclerotic plaques and terminal arterioles, which define the systemic vascular resistance. At the reflection sites, the reflected waves are generated and travel towards the heart, superimposing on the forward waves. Because the pressure wave's velocity is very fast, the backward wave usually superimposes on the same forward wave generating it. As a consequence of this superimposition, the blood pressure wave as measured in the PPG signal is a combination of the forward pressure wave, moving from the heart to the extremities, and the backward pressure wave, reflected back towards the heart. The pulse wave velocity is very fast, resulting in the imposition of the reflective wave to be near instantaneous. The time delay between systolic and diastolic peaks is related to the transit time of pressure waves from the root of the subclavian artery to the apparent site of reflection and back to the subclavian artery and this path length may be assumed to be proportional to the height of the subject. In the case where the elasticity of the aorta at the reflecting site is good and the arterial stiffness is low, the backward wave will arrive at the upper limb at a slower rate. However, in the case that the arterial stiffness of the aorta at the reflecting site is high, for instance if the subject is elderly, the blood stroke will be reflected back at a quicker rate due to the reduced elasticity. This implies that older subjects will have shorter systolic peak (S_{y}) to diastolic peak (D_{y}) time and higher stiffness index in the pulse waveform when compared to younger subjects. This corresponds to the observed increase in the stiffness index as a function of time, as reported in literature, and illustrated by Equation 6.

$$Stiffness index = \text{height/} ( {D}_{y} - {S}_{y} ) \qquad [ Equation 6 ]$$

The pulse width, which is another feature of interest, is defined as the width of the pulse at half the height of the systolic pulse in the PPG pulse waveform. The pulse width has been suggested to positively correlate with the systemic vascular resistance better than the systolic amplitude. See e.g. Awad, Aymen A., Ala S. Haddadin, Hossam Tantawy, Tarek M. Badr, Robert G. Stout, David G. Silverman, and Kirk H. Shelley. “The relationship between the photoplethysmographic waveform and systemic vascular resistance.” Journal of clinical monitoring and computing 21, no. 6 (2007): 365-372. The systemic vascular resistance describes the resistance to blood flow throughout the entire system vasculature. The greatest amount of resistance comes from arterioles and small arteries which have a very thick tunica media and tunica adventitia.

Based on the Hagen-Poiseuille law, there are three key determinants of vascular resistance: blood viscosity, vessel length and vessel radius. The total peripheral resistance is almost entirely due to changes in the diameter of arterioles and small arteries. The smaller the radius of the vessel, the larger resistance will be for blood flow. In a normal vessel, the smooth muscle around it can modulate its diameter to contract (vasoconstriction) or to expand (vasodilation), so the blood pressure and flow can be modulated with the vascular resistance. A PPG pulse represents the vessel volume variation as the blood stroke goes through the wrist/fingertip small arteries. For a blood pulse with a certain volume, the small arteries with a good elastic wall and muscle are easier to expand, like the aorta with good elasticity. Therefore, the amplitude of a pulse is higher and the width is relatively shorter and the pulse velocity to go through arteries is slower. In contrast, as the elasticity of vessel wall and muscle reduces and the arteries become stiff, the diameter of small arteries becomes harder to modulate. Therefore, the vascular resistance increases and the blood stroke has to go through the vessels with an increasing velocity. Finally, the pulse will prefer to broaden its width instead of increasing its amplitude. When blood vessel walls are damaged, their ability to dilate or constrict to adapt to hemodynamic changes becomes impaired. This damage often leads to too high resistance in that vessel, causing further damage to the vessel, hypertension or preventing the flow of blood to that vascular territory.

As was mentioned in the discussion regarding SEVR there are two actions in the systolic phase, the first being the Isovolumic contraction in the left ventricle, and the second being the ventricular ejection into the aorta and the aorta expansion and storage of the majority of the stroke volume. When interpreting the APG pulse waveform, the a wave represents the acceleration of the ventricle ejection, whereas the b wave predominantly represents the “buffer” reduction acceleration. Since there is a time delay between the heart and the point of detection, the systolic increasing period matches the T peak in the ECG signal. Therefore, the a wave corresponds to the early moment during the ventricular ejection, after isovolumetric contraction. The size of the a wave corresponds with the strength of the heart muscle. The larger the a wave, the stronger the heart muscle. In contrast, the b wave is influenced by the aorta and arterial stiffness. In the case that the aorta and arterial stiffness is high, the amount of stroke volume in storage in the aorta during the systolic phase is low, while most of the stroke volume is pushed directly toward the peripheral vessels. This suggests that the aorta pressure function is reduced and the absolute value of the b wave decreases. An increase in the b/a ratio indicates that the aorta and arterial stiffness increases, alluding to the presence of hypertension in the subject. Hypertensive subjects have been observed with higher (less negative) b/a ratio, as discussed in Zhang, Yahui, Zhihao Jiang, Lin Qi, Lisheng Xu, Xingguo Sun, Xinmei Chu, Yanling Liu, Tianjing Zhang, and Stephen E. Greenwald. “Evaluation of cardiorespiratory function during cardiopulmonary exercise testing in untreated hypertensive and healthy subjects.” Frontiers in physiology 9 (2018): 1590.

Anomaly detection in time-series data is a well-studied phenomenon to which a large range of techniques have been applied. Most recently, with larger volumes of Internet of Things (IoT) data recorded on human subjects and advances in computer capability, an opportunity has opened up for training more sophisticated models, such as deep neural networks to detect physiological anomalies on time-series data. Anomaly detection can include elements of unsupervised, supervised and semi-supervised machine learning to characterize areas in the input feature space that correspond to normal physiology, known disease states or conditions as well as unknown, but more likely abnormal regions falling outside the scope of normal physiology. There is some literature available on the training of supervised deep neural networks to detect specific conditions, like atrial fibrillation (Afib), hypertension, heart rate and other biometrics from PPG data. As an example of deep neural networks for PPG signal data, work performed by the research group of Andrew Ng proposed that a 50 layer neural network model, referred to a ResNeXt, can directly analyze the PPG pulse waveform and achieve a test AUC of 95% for atrial fibrillation detection See Shen, Yichen, Maxime Voisin, Alireza Aliamiri, Anand Avati, Awni Hannun, and Andrew Ng. “Ambulatory Atrial Fibrillation Monitoring Using Wearable Photoplethysmography with Deep Learning.” In Proceedings of the 25th ACM SIGKDD International Conference on Knowledge Discovery & Data Mining, pp. 1909-1916. 2019. Recent literature has presented the potential of deep neural networks in PPG pulse waveform analysis.

In an aspect, the current invention is based on using the PPG pulse waveform and its derivatives, in combination with anomaly detection approaches, for the identification and monitoring of healthy and unhealthy states on an integrated IoT platform spread over multiple devices including, but not limited to, wrist worn wearable devices, cellular smartphone, and cloud computing infrastructure for the purposes of storage, computing and communication with users or interested third parties.

Although anomaly detection is a broad field, a specific implementation of the current invention is used as a concrete example of the methodology. Convolutional neural networks (CNNs) are leveraged as a specific method commonly used for the analysis of visual imagery and have been used in applications such as image recognition, image classification, image segmentation, natural language processing, and brain-computer interfaces. CNNs are also commonly referred to as shift invariant, or space invariant artificial neural networks (SIANN). This naming is derived from the shared weight architecture of the convolution kernels that slide along the input features in a similar fashion as the event and cycle windows described for the two-moving-average method and provide translation equivariant responses as feature maps. CNNs are regularized versions of multilayer perceptron where each neuron in a single layer is connected to all the neurons of the following layer in the multilayer perceptron. It has the drawback that this full connectivity is prone to overfitting of data and relies on the addition of regularization to prevent such overfitting. These regularization methods may include, but are not limited to, taking advantage of the hierarchical pattern in the data and assembling patterns of increasing complexity by using smaller and simpler patterns embossed in the CNN filters.

In an aspect, CNNs are on the lower extreme when it comes to the connectivity and complexity of neural networks and use relatively little pre-processing in comparison to other image classification algorithms, making it ideal for real-time monitoring applications such as is expressed in the current invention. The network learns to optimize the kernels through automated learning as opposed to hard engineering the kernels, making it independent from prior knowledge and human intervention to extract features. CNNs are generally used as part of supervised learning methods, meaning that a CNN may be constructed by training the network on data with specific labels associated with the data, for instance in this case PPG signal data of healthy and unhealthy participants may be presented to the network along with the indication that the participant is healthy or, in the case that the participant is unhealthy, the disease or condition that the participant is suffering from. This allows the CNN to extract features that will highlight the differences in the different health states.

For instance, consider the application of CNNs in the analysis of the PPG pulse waveform for the identification of Afib, several distinct features in the pulse waveform correspond to the health states of the AFib and non-AFib participants, as discussed in Shen, Yichen, Maxime Voisin, Alireza Aliamiri, Anand Avati, Awni Hannun, and Andrew Ng. “Ambulatory atrial fibrillation monitoring using wearable photoplethysmography with deep learning.” In Proceedings of the 25th ACM SIGKDD International Conference on Knowledge Discovery & Data Mining, pp. 1909-1916. 2019. In an aspect, feature extraction through the use of such a CNN setup generates a latent space representation, or manifold, that may be constructed during training on the pulse waveform data. Training can be done in a supervised configuration, semi-supervised or unsupervised configuration. For a supervised configuration, the CNN is part of a classification network trained on pulse waveform datasets from individuals that are normal, individuals that are on different parts of the wellness spectrum as well as individuals with a diagnosis of one or more cardiovascular conditions or diseases. For an unsupervised configuration, the CNN can be trained as part of a variational autoencoder architecture or a generative adversarial network (GAN), both of which are trained on unlabeled data.

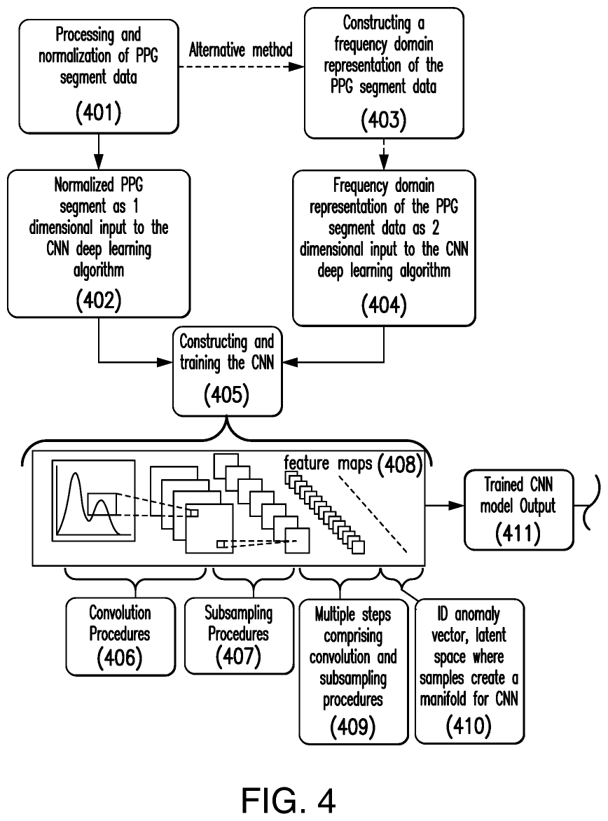

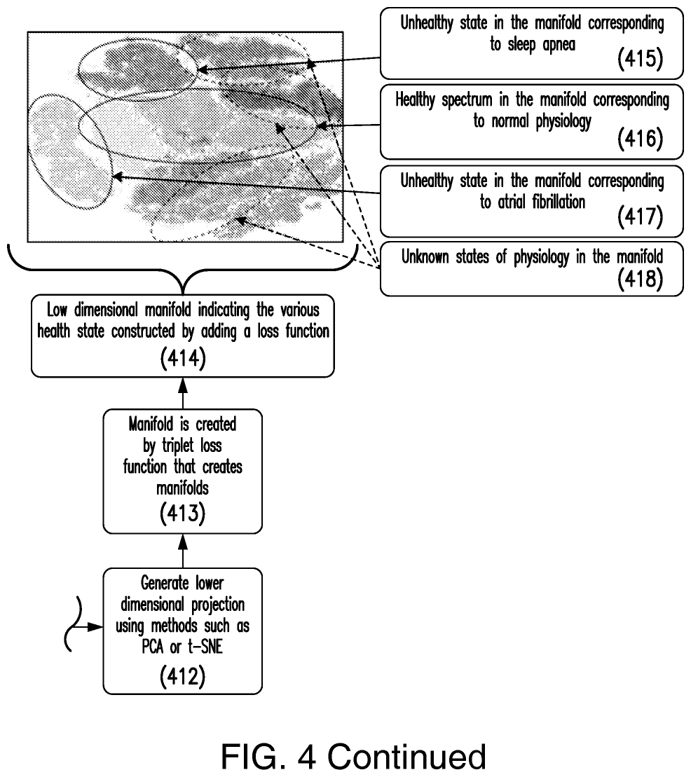

Once trained, the CNN can be used to generate outputs in its latent space manifold (410) (see FIG. 4), the output layer that precedes the classification stage, which are outputs that have not yet been converted into a categorical classification. As a next step toward anomaly detection, a technique for reducing the outputs in the latent space manifold is applied, typically a technique such as PCA (principal component analysis) or t-SNE (t-distributed stochastic neighbor embedding) (412-413). The output of this step would be a low dimensional (e.g. two dimensional (2D) or three dimensional (3D)) visualizable map (414) and a set of domains in this space is subsequently defined in line with where available datasets show up when processed by the CNN and dimension reduction technique (PCA or t-SNE). Specific domains would correspond to specific conditions or disease states, for instance data from persons diagnosed with sleep apnea (415) or atrial fibrillation (417), normal health states for individuals without diagnosis of chronic disease spanning age and sex demographics (416) and anomalous health states, which fall outside of the disease states and normal health states (418).

To provide more concrete information on this process of creating a low dimensional space for classifying data in this manner, the t-SNE embedding method is elaborated upon here. A t-SNE method may be allied to the last convolutional layer output vectors in high-dimension, for instance a CNN output of 1024 dimensions, to transform the CNN outputs into two dimensional data points in a way that similar samples may be gathered into a cluster of nearby points, whereas dissimilar data points may be modeled to be other points or clusters further away. This results in the formation of clusters on the manifold with each cluster corresponding to a specific health state. Considering an N amount of PPG signals that may be processed and therefore an N amount of CNN high-dimensional objects as outputs of the PPG signals (x₁, . . . , x_{N}), a t-SNE method may be used to calculate a probability (pᵢⱼ) that indicates the similarity between any two objects xᵢ and xⱼ. This may be done using Equation 7 and Equation 8.

$${p}_{i / j} = exp ( - {‖ {x}_{i} - {x}_{j} ‖}^{2} / 2 {σ}_{i}^{2} ) / {\sum }_{k \neq i} exp ( - {‖ {x}_{i} - {x}_{k} ‖}^{2} / 2 {σ}_{i}^{2} ) \qquad [ Equation 7 ]$$

$${p}_{ij} = ( {p}_{i / j} + {p}_{i / j} ) / 2 N \qquad [ Equation 8 ]$$

Equation 7 evaluates how similar the object xⱼ is to xᵢ by calculating the conditional probability pᵢⱼ that xᵢ and xⱼ are close to one another in proportion to their probability density under a Gaussian distribution centered at xᵢ. Furthermore, the bandwidth of the Gaussian kernels (σᵢ) may be set in such a way that the conditional distribution is equal to a predefined perplexity. The aim of the t-SNE technique is to learn a d-dimensional map (y₁, . . . , y_{N}) that reflects the similarity in probabilities pᵢⱼ that two objects are similar. Therefore, the similarities of two objects are measured (qᵢⱼ) between any two points, yᵢ and yⱼ, on the manifold which may be done using Equation 9.

$${q}_{ij} = {( 1 + {‖ {y}_{i} - {y}_{j} ‖}^{2} )}^{- 1} / {\sum }_{k} {\sum }_{l \neq k} {( 1 + {‖ {y}_{k} - {y}_{l} ‖}^{2} )}^{- 1} \qquad [ Equation 9 ]$$

Equation 9 may be used to measure the similarities (qᵢⱼ) between 2 low-dimensional points (yᵢ and yⱼ) to allow for dissimilar objects to be modeled apart from one another in the manifold. The location of each point yᵢ in the manifold may be determined by minimizing the non-symmetric Kullback-Leibler divergence of the distribution P from the distribution Q as illustrated in Equation 10. This results in a low-dimensional representative manifold that reflects the similarities between high-dimensional inputs.

$$KL ( P ‖ Q ) = {\sum }_{i \neq j} {p}_{ij} log ( {p}_{ij} / {q}_{ij} ) \qquad [ Equation 10 ]$$

In an aspect, the addition of a loss function such as, but not limited to, triplet loss, mean square error loss, and cross entropy loss may be used to organize the manifold and to create continuity in the feature set classes. A loss function, also referred to as a cost function, maps the values of one or more features or predictions onto a real number, which intuitively represents a cost or loss associated with the placement of that feature, or set of features within the manifold. When performing an optimization, the objective would be to minimize the loss function, ensuring that the feature set describing a specific health state is not assigned incorrectly in the manifold. For instance, consider using triplet loss as the loss function, the triplet may be formed by drawing an anchor input (A), a positive input (P) that describes the same health state as the anchor, and a negative input (N) that describes a different health state than the anchor. The inputs may then be run through the network and the outputs may be used in the loss function. This may be done by describing the loss function [symbol] (A, P, N) as a Euclidean distance function as shown in Equation 11. The Euclidean distance function may then be used in the cost function [symbol] as detailed in Equation 12.

$$ℒ ( A , P , N ) = max ( {‖ f ( A ) - f ( P ) ‖}^{2} - {‖ f ( A ) - f ( N ) ‖}^{2} + a , 0 ) \qquad [ Equation 11 ]$$

$$𝒥 = {\sum }_{i = 1}^{M} L ( Ai , Pi , Ni ) \qquad [ Equation 12 ]$$

In Equation 11, a refers to a margin between the positive and negative pairs, whereas f refers to an embedding function. The addition of a loss function allows for organizing the data points distributed on the manifold to be classified according to the various health states present in the manifold. The organization of the manifold will result in areas on the manifold corresponding to a known healthy state and known unhealthy states whereas areas within the manifold that cannot be placed within the organized areas, due to, for instance, a lack of training data diversity, may contain unknown health states.

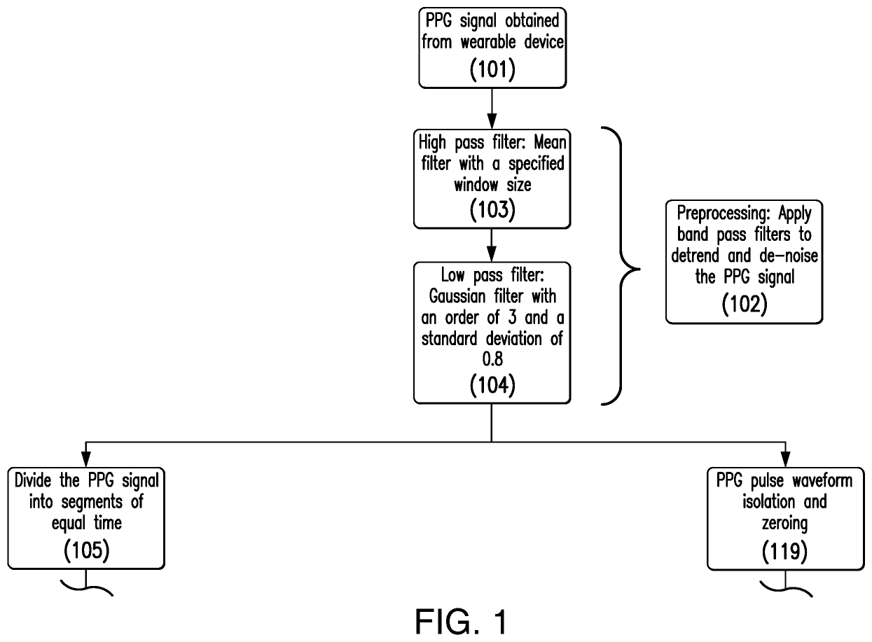

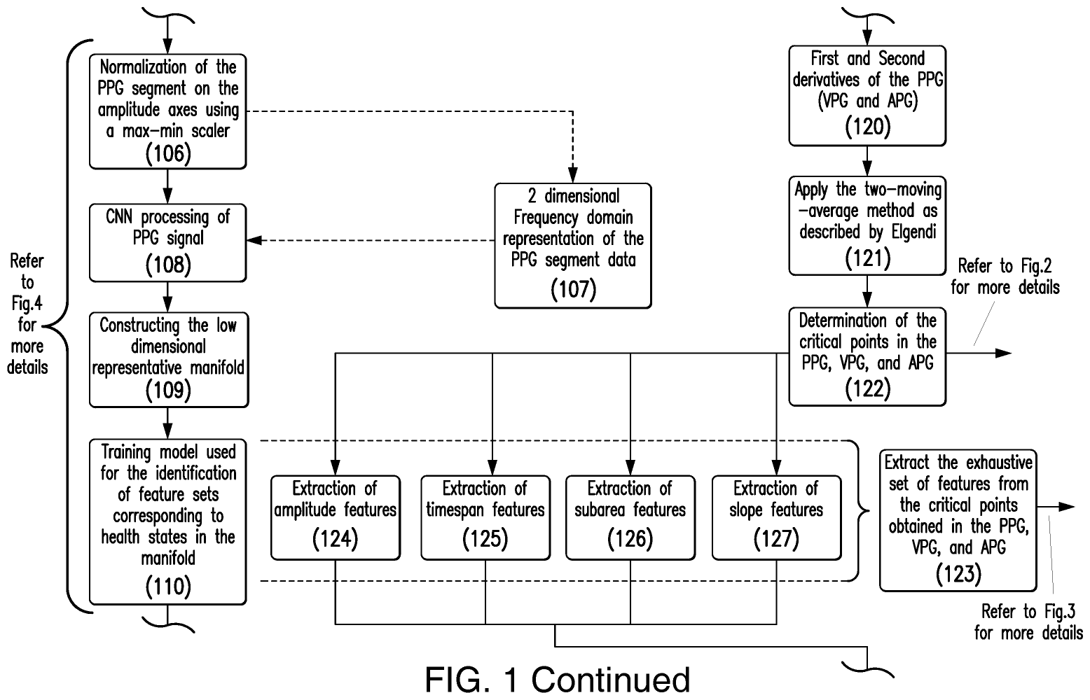

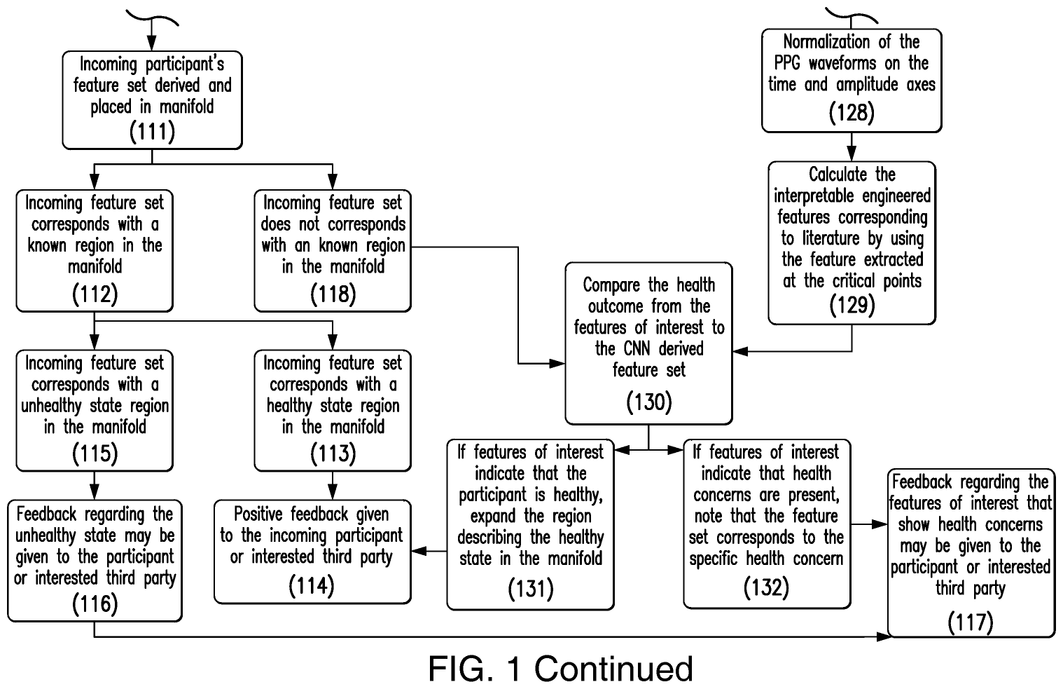

In an aspect of the present invention as shown in FIG. 1, the PPG signal may be obtained from wearable devices (101) such as, but not limited, to fingertip pulse oximeter, earlobe pulse oximeter as well as in smart wearable devices such as wrist worn devices and smart rings. The required preprocessing may be applied to the PPG signal (102) such as, but not limited to, detrending and de-noising using methods such as, but not limited to the Butterworth filter, the inverse Chebyshev filter, or other high-pass and low-pass filters commonly understood to those known in the arts, to improve signal quality. In some respects, knowing the base frequency of the PPG signal may assist in pre-processing or in de-noising the signal. For the description of this exemplary embodiment of the invention a mean filter may be used with a sample window of, for instance, 50 as a high pass filter (103) to only allow signal data above 0.5 Hz to pass through the filter. This may be followed by a Gaussian filter with, for instance, an order of 3 and a standard deviation of 0.8 as a low pass filter which only allows a signal of less than 6 Hz to pass through the filter (104).

The detrended and de-noised PPG signal may be used for automated feature extraction using a CNN setup, as well as for the conventional extraction of the interpretable engineered one-dimensional (1D) features including, but not limited to crest time, SEVR, ejection duration, large artery stiffness index, small artery resistance and features related to hypertension, which has been reported in literature. For automated feature extraction using a CNN setup according to an aspect, the PPG signal is divided up into equal timespan segments (105), for instance 30 second segments. These PPG segments may then be normalized by using a min-max scaler to scale the amplitude of the PPG segment to range from 0 to 1 (106) to generate a one-dimensional representation of the PPG signal data, described in more detail in FIG. 4. Alternatively, a two-dimensional input dataset may be generated from the PPG signal by generating a frequency domain representation of the PPG signal such as, but not limited to, Fourier spectrum, Lomb-Scargle periodogram or by cardiopulmonary coupling (107) described in more detail in FIG. 4.

A CNN is constructed for the classification of the health states of participants (108). The high dimensional output from the CNN setup may be used along with methods such as t-SNE and triplet loss to automatically construct a low dimensional representative manifold with feature sets corresponding to the different possible health states present in the participants (109). This training setup allows for the allocation of participants with unknown health states to be classified in the manifold to identify any underlying health risks that the participants may have (110). Therefore, the feature set derived for an incoming participant (e.g. a user) may be derived using the CNN setup and the feature set may be placed on the manifold to evaluate the health state of the incoming participant (111).

In the case where a participant's data corresponds to a feature set described in the manifold (112) and the feature set corresponds with a healthy state as laid out in the manifold (113) then positive feedback may be reported to the participant or interested third party indicating that the participant is healthy (114).

In the case where a participant corresponds to a feature set described in the manifold that deviates from the healthy feature set, as expressed in the manifold, allocation of the feature set in the manifold may aid to identify which health concerns may be present in the participant.

In the case where the feature set of the participant corresponds with the feature set of a known unhealthy state (115), as expressed in the manifold, then the participant, or interested third party, may be given feedback on the specific unhealthy state that corresponds with the participant's feature set (116).

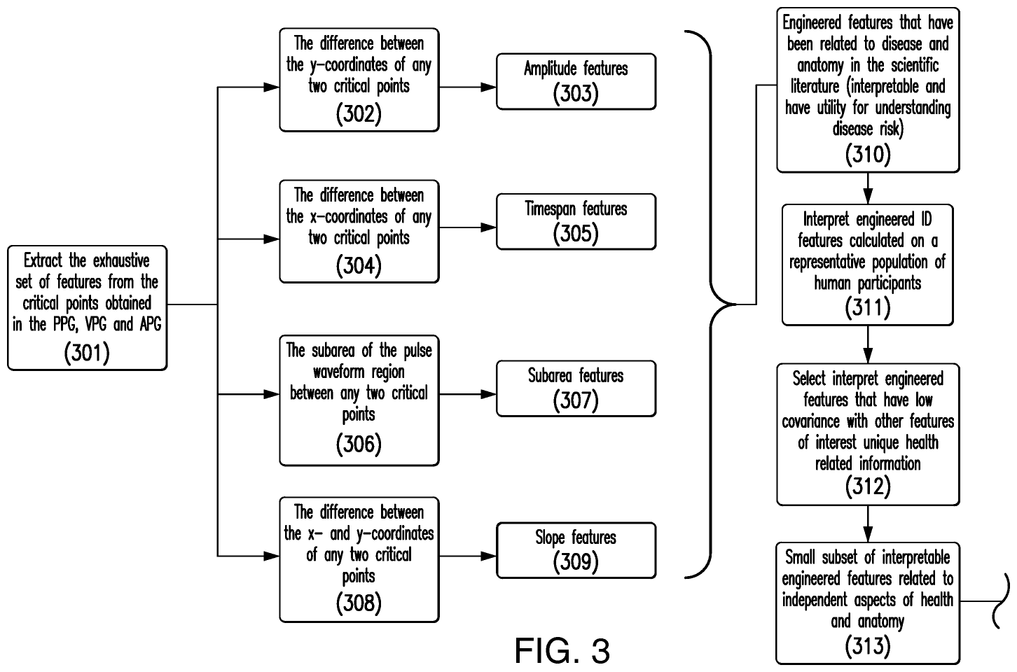

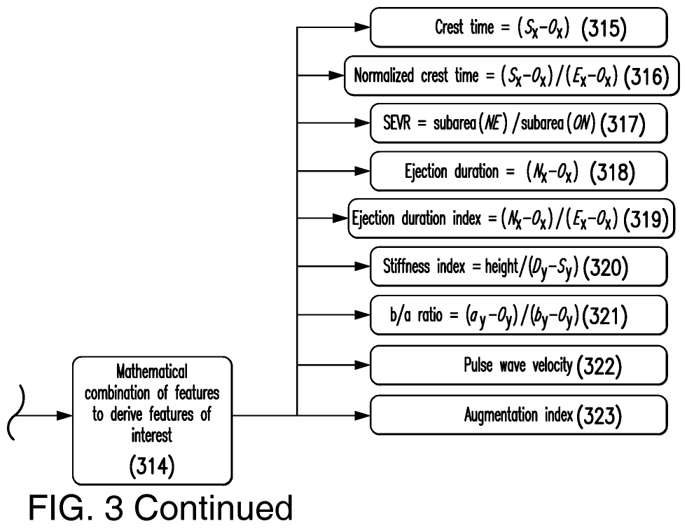

Feedback pertaining to the interpretable engineered one-dimensional (1D) features, as derived through the conventional analysis of the PPG signal as shown in FIG. 3 may also be given to the user or interested third parties (117).

In the case where the feature set does not correspond to any of the feature sets describing the known healthy or unhealthy states expressed in the manifold (118) and corresponds to an unknown region in the manifold, then the feature set corresponding with this participant may be classified as an unknown health state of concern within the manifold. The resulting feature set may then be compared to the interpretable engineered one-dimensional (1D) features as derived through the conventional means of pulse waveform analysis.

In another aspect, the PPG signal may be obtained from wearable devices (101). The required preprocessing may be applied to the PPG signal (102) to improve signal quality for the identification of the critical points. Similarly, to the above-mentioned exemplary embodiment of the invention, a mean filter is used with a sample window of 50 as a high pass filter (103) followed by a Gaussian filter with an order of 3 and a standard deviation of 0.8 as a low pass filter (104). The individual pulse waveforms may be isolated from the PPG signal (119), and may be followed by zeroing of the pulse waveform. This is followed by the determination of the first and second derivatives of the pulse waveforms to obtain the pulse waveforms of the PPG, VPG and APG respectively (120). Methods such as, but not limited to, the two-moving-average method, as described by Elgendi may be applied to each PPG, VPG and APG pulse waveform (121) for the determination of the critical points associated with the PPG, VPG and APG pulse waveforms (122).

The critical points may be used for the determination of an exhaustive set of features (123), derived from the differences in the positions of the critical points. The amplitude (124), timespan (125), subarea (126) and slope features (127) may be derived to generate this exhaustive set of features, and these features may be normalized (128) and used to derive the interpretable engineered one dimensional (1D) features (129) including, but not limited to crest time, SEVR, ejection duration, large artery stiffness index, small artery resistance and features related to hypertension, which has been reported in literature as illustrated in FIG. 3.

As laid out in the previous exemplary embodiment of this invention, the CNN setup may obtain feature sets of an unknown health state that does not compare to known healthy or unhealthy states described in the manifold (118). The interpretable engineered one dimensional (1D) features may be derived for participants that may have a feature set of an unknown health state to determine whether there are underlying health concerns present in the participant (130) and will be discussed in more detail below. In the case that the interpretable engineered one dimensional (1D) features show that the participant is healthy, the comparison information may be used to improve the understanding of the CNN manifold by expanding the accepted feature set that corresponds to the healthy state (131). Furthermore, positive feedback may be reported to the participant or interested third party indicating that the participant is healthy (114). In the case that the interpretable engineered one dimensional (1D) features shows that underlying health concerns may be present, then the specific interpretable engineered one dimensional (1D) features may be noted as being associated with the specific feature set in the manifold as obtained by the CNN setup (132), and the participant or interested third party may be notified that the participant has the specific interpretable engineered one dimensional (1D) features which show health concerns (117).

In an aspect, the conventional method of analyzing the PPG signal for deriving the critical points may be used to determine the interpretable engineered one dimensional (1D) features for comparison to the automatically derived CNN features. The PPG pulse waveform (201) may be used for determination of five critical points, as shown in FIG. 2. The first derivative and second derivatives of the PPG signal may be derived to obtain the VPG (202) and APG (203) signals for the determination of the remaining eight critical points.

The critical points corresponding to the start of the PPG pulse waveform, O (204), the systolic peak, S (205), the dicrotic notch, N (206), the diastolic peak, D (207), and the end of the PPG pulse waveform, E (208) may be determined from the PPG signal. The critical points corresponding to the maximum positive velocity in the systolic phase, w (209), the minimum negative velocity in the systolic phase, y (210), and the maximum positive velocity in the diastolic phase, z (211) may be determined from the VPG signal. The critical points corresponding to the a to e waves in the APG signal, referred to here as critical point a (212), critical point b (213), critical point c (214), critical point d (215), and critical point e (216) may be determined from the APG signal.

In an aspect, the two-moving-average method as published by Elgendi may be used to identify regional blocks in the PPG, VPG and APG pulse waveforms for the determination of the location of the critical points. The block regions that contain each of the specific critical points may be identified and the maximal- or minimal point in each of the block regions of interest will correspond to the critical point associated with that specific block region. Using this method, the x- and y-coordinates of each critical point for a given pulse waveform may be determined.

For the comparison of the interpretable engineered one dimensional (1D) features, as derived through the conventional analysis of the PPG signal, with the CNN derived feature set, a clear understanding of these features and their classifications of healthy and unhealthy states are required. The ability to obtain the x- and y-coordinates for each critical point for each pulse waveform, or representative pulse waveform, in a series of pulse waveforms allows for the derivation of further features to describe the PPG signal.

In an aspect, the critical points derived from the PPG, VPG and APG respectively may be used for the extraction of an exhaustive set of features (301), as shown in FIG. 3. The difference between the y-coordinates of any two critical points (302) may be calculated to derive the amplitude features corresponding to the pulse waveform (303). The difference between the x-coordinates of any two critical points (304) may be calculated to derive the timespan features corresponding to the pulse waveform (305). The PPG, VPG and APG pulse waveforms may be divided into subsections between the different critical points where any two critical points can generate an area under the pulse waveform subsection (306). Each sub-area may or may not be normalized by the total area under the entire pulse waveform and each sub-area may be integrated using methods such as, but not limited to, numerical integration, or integration methods based on the trapezoidal rule, to derive subarea features (307). The difference between the x- and y-coordinates of any two critical points (308) may be calculated to derive the slope features corresponding to the pulse waveform (309).

Within literature there are several publications focused on deriving health related and interpretable engineered features from the exhaustive set of amplitude, timespan, subarea and slope features (310). These interpretable engineered one dimensional (1D) features have been related to disease and anatomy in the scientific literature and are both interpretable and have a utility in understanding the risk of disease development. The health related and interpretable features may be calculated for a representative population of human participants (311), and methods such as, but not limited to dimensionality reduction techniques for setting up covariance matrices, or PCA may be used to remove interpretable engineered one dimensional (1D) features or to select engineered one dimensional (1D) features that hold unique health related information (312).

Considering that a covariance matrix may be constructed, the covariance matrix generalizes the notion of variance to multiple dimensions. This allows for the elimination of interpretable engineered one dimensional (1D) features with high covariances to reduce the set of engineered features to only those features that are not covariant with the other features to identify features that hold unique information regarding cardiovascular health and anatomy. In an aspect, PCA may be used for the elimination of features that do not contain unique health related information, which is the process of computing the principal components of a dataset and using these principal components to perform basis transformation on the data. This results in a small subset of interpretable engineered one dimensional (1D) features that relates to independent aspects of health and anatomy (313). The mathematical equations for the calculation of this small subset of features are made available in the scientific literature and these mathematical equations may be used to derive the interpretable engineered features (314). Health related features such as, but not limited to, crest time (315), normalized crest time (316), SEVR (317), ejection duration (318), ejection duration index (319), stiffness index (320), the b/a ratio (321), pulse wave velocity (PWV) (322), and augmentation index (323), as well as interpretable engineered one dimensional (1D) features that may become available in future, may be derived through the conventional analysis of the PPG signal.

In an aspect, the PPG signal may be used to generate an input data sequence for the CNN deep learning algorithm. A discussion follows of some data preparation methods that may need to be considered. A series of PPG pulse waveforms are collected, for instance 3000 pulses, which may then be sorted and concatenated along the timeline, resulting in a sequence with 3000 pulses. This pulse sequence may then be divided into a series of smaller sequences of fixed length, for instance 100 smaller sequences of 30 pulses per sequence. These smaller sequences may then iteratively be fed into the CNN for training. The PPG signal can also be divided up into equal timespan 30 second segments (105).

After detrending and de-noising, the PPG segments may be normalized by using a min-max scaler to scale the amplitude of the PPG segment to range from 0 to 1 to generate a one dimensional representation of the PPG signal data, whereas the timescale remains un-normalized (401), as shown in FIG. 4. This one dimensional data series will act as the input dataset to the CNN (402). In an aspect, an alternative input dataset may be generated from the PPG signal by generating a frequency domain representation of the PPG signal such as, but not limited to, Fourier spectrum, Lomb-Scargle periodogram or by cardiopulmonary coupling as a two dimensional representation of the pulse waveform (403). A CNN deep learning algorithm may then be trained on the frequency domain representation of the PPG signal rather than the PPG signal input for the construction of the CNN manifold for the identification of health states (404). Regardless of whether the PPG signal or a frequency domain representation of the PPG signal are used as input data, the input data may be used to construct the CNN and train the CNN to derive a feature set for the identification of a user's health state (405).

To train the CNN a convolution procedure is performed on the input data (406), followed by a subsampling procedure (407). Each procedure generates a series of feature maps (408) correlating the features according to the health states of the users. Multiple steps comprising convolution may be included and subsampling steps may be required before an adequate CNN may be derived (409). The latent space output from a fully trained CNN model (410) in addition to the classification output (411) may then be used to construct a low dimensional manifold by incorporating methods such as, but not limited to, principal component analysis or t-SNE to generate lower dimensional projections on the manifold (412). Furthermore, using a loss function, for instance triplet loss (413) allows for the identification of the various health states in the manifold which allows for the mapping of the various health states on the manifold (414).

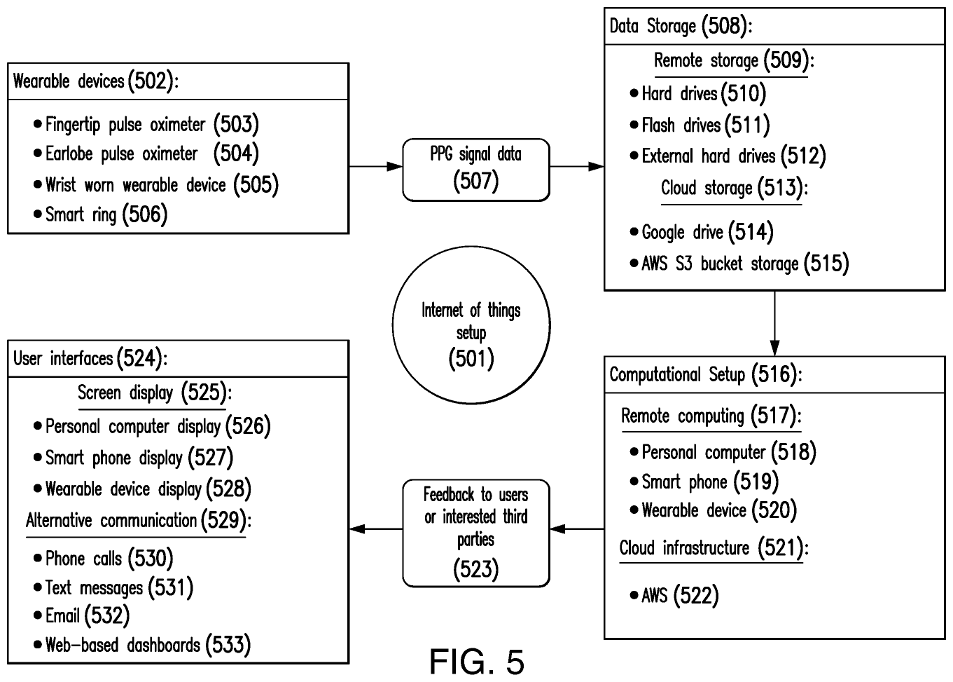

In an aspect, the system is implemented on an IoT setup (501) as shown in FIG. 5, which includes a wearable device for the monitoring of the PPG signal (502) such as, but not limited to, fingertip pulse oximeter (503), earlobe pulse oximeter (504), wrist worn wearable device (505), and smart ring (506). The PPG signal data obtained from the wearable devices (507) will require storage (508), which can include remote data storage devices (509) such as, but not limited to hard drives (510), flash drives (511) or external hard drives (512), and cloud infrastructure for the storage of data (513) such as, but not limited to Google drive (514) or Amazon web services (AWS) S3 buckets (515).

The computational calculations will be performed on a setup (516) reliant on remote computing (517) on devices such as, but not limited to, personal computers (518), smart phones (519), and wearable devices (520). Cloud infrastructure may be used for the implementation of computational calculations (521) using services such as, but not limited to, AWS (522). For the purposes of giving feedback (523) to users, or interested third parties, regarding the health status of users, several user interfaces may be utilized (524) including devices with screen displays (525) such as, but not limited to desktop computer displays (526), smartphone display (527) and wearable device display (528) for reporting feedback to users. Feedback regarding the health status of users may also be given to the users, or interested third parties, using alternative methods of communication (529) such as, but not limited to phone calls (530), text messages (531), email communication (532) or through a web-based dashboard (533).

In an aspect, the current invention provides a means of using wrist-worn wearable devices to detect early changes in cardiovascular health. A monitored user that has a stable crest time feature, which initially remains a stable value, will be categorized by the neural network (411) employed to analyze the user's features as being part of a ‘normal’ category (416) that the model was trained on. However, if the crest time of the user starts changing due to early signs of heart failure as a result of disease that affects the heart muscle, the crest time feature would gradually increase along with other cardiovascular features derived from the wearable, such as heart rate and heart rate variability. This change will follow a pattern that moves the user from the healthy part (416) of the lower dimensional manifold (created for the model using t-SNE or PCA) (412) toward the direction on the lower dimensional manifold (412), associated with heart failure. When this becomes a statistically significant deviation compared to the deviations seen for healthy users, the system generates an alert that can be sent to care providers (523) to perform further follow up and determine the cause of this observation.

Further, the system can provide an all-round means for detecting cardiovascular changes that are statistically significantly distinguishable from changes seen in training data on a healthy cohort (405). All-round in this context, means that when the PPG derived pulse waveform data that enters the convolutional neural network layer described in FIG. 4, is projected down to the 2 dimensional plane created by t-SNE or PCA (414), and a user either moves within that 2D plane toward the outer edge of the healthy area (416) or crosses outside of the healthy area (416), the system triggers an alert and shares the interpretable features of the pulse waveform (523), for example heart muscle strength via crest time, coronary perfusion via SEVR, large artery health via augmentation index, with care providers for further investigation.

In an exemplary embodiment of the invention, consider that the PPG signal of a user with unknown cardiovascular health status is obtained. The PPG signal is fed into the CNN setup to evaluate the health status of the user. Consider then that the result obtained from the CNN analysis of the PPG signal indicates that the PPG signal of the user does not correlate with a healthy state, however, the PPG signal of the user cannot be placed within a known unhealthy state as mapped on the CNN manifold. Through the conventional analysis of the PPG signal of the user, the large artery stiffness index, PWV and the augmentation index have been shown to be higher than normal. Given the results obtained through the conventional analysis of the pulse waveform, a literature search may be conducted or a medical professional may be consulted which concludes that these trends are consistent with a patient suffering from hypertrophic cardiomyopathy (HCM), which is a thickening of the heart muscle with a genetic origin. Feedback may then be given to the user and interested third parties, such as a medical professional regarding the health concern and the manifold of the CNN may be extended to include this heath state. Furthermore, the health state of the user may be monitored over time and reported to the user or their medical practitioner regarding the progression of the disease so that preventative interventions may be performed.

In an aspect, consider that the PPG signal of a user with unknown cardiovascular health status is obtained. The PPG signal is fed into the CNN setup to evaluate the health status of the user and the resulting feature set corresponds with an unknown region in the CNN manifold. By calculating the interpretable and physiologically relatable features of the pulse waveform, that characterize individual anatomical aspects of health, such as large vessel health, small vessel health, blood pressure, myocardium health (discussed earlier), it might be possible to classify anomalies generated on this individual as potentially disease related due to reduced myocardium health pulse waveform features being associated with the anomaly.

The user, or an interested third party such as a medical practitioner, may be notified of the health status of the user. Consider that the medical practitioner may recommend lifestyle changes to the user and therefore has an interest in continuously monitoring the heart health state of the user as derived by the pulse waveform anomaly detection system, to evaluate the efficacy of the recommended lifestyle changes. The results from the pulse waveform anomaly detection system may be made available to the medical practitioner through a web-based dashboard which reports a summary of the health state of the user over time, allowing for remote monitoring of the user. Similarly, the medical practitioner may have an interest in remotely monitoring multiple patients using such a web-based dashboard. This will enable the medical practitioner to frequently investigate the health states of multiple patients and make changes to their medical care, when required, without the need for personal interaction with each patient.

Although several aspects have been disclosed in the foregoing specification, it is understood by those skilled in the art that many modifications and other aspects will come to mind to which this disclosure pertains, having the benefit of the teaching presented in the foregoing description and associated drawings. It is thus understood that the disclosure is not limited to the specific aspects disclosed hereinabove, and that many modifications and other aspects are intended to be included within the scope of any claims that can recite the disclosed subject matter.

It should be emphasized that the above-described aspects are merely possible examples of implementations, merely set forth for a clear understanding of the principles of the present disclosure. Any process descriptions or blocks in flow diagrams should be understood as representing modules, segments, or portions of code which comprise one or more executable instructions for implementing specific logical functions or steps in the process, and alternate implementations are included in which functions may not be included or executed at all, can be executed out of order from that shown or discussed, including substantially concurrently or in reverse order, depending on the functionality involved, as would be understood by those reasonably skilled in the art of the present disclosure. Many variations and modifications can be made to the above-described aspect(s) without departing substantially from the spirit and principles of the present disclosure. Further, the scope of the present disclosure is intended to cover any and all combinations and sub-combinations of all elements, features, and aspects discussed above. All such modifications and variations are intended to be included herein within the scope of the present disclosure, and all possible claims to individual aspects or combinations of elements or steps are intended to be supported by the present disclosure.

What is claimed is:

1. A method for identifying a health status of a user using an IoT setup of interconnected electronic devices and sensors, the method comprising:
(a) collecting a user's PPG signal using a wearable device;
(b) preprocessing of the PPG signal for a conventional analysis of the PPG signal and extraction of critical points and interpretable engineered one dimensional (1D) features such as, but not limited to, crest time, SEVR, ejection duration index, large artery stiffness index, small artery resistance and features relating to hypertension;
(c) using an anomaly detection system, exemplified by a convolutional neural network trained on pulse waveform data, to produce a feature vector in a latent space;
(d) using a dimension reduction method to construct a low dimensional representation (two-dimensional or three-dimensional) of the feature vector;
(e) labeling sections of the two-dimensional/three-dimensional space as corresponding to healthy, specific condition or unknown based on the class assigned to the PPG signal by the anomaly detection system;
(f) creating interpretable engineered 1D features that refer to specific physiological processes associated with health risk;
(g) using the interpretable engineered features together with the healthy/disease/unknown output of the anomaly detection system, to resolve ‘unknown’ anomalies as being healthy or unhealthy based on whether the interpretable engineered features have 1D values associated either health or specific condition; and
(h) providing feedback to the user, or to a third party, regarding the health states of the user where in the case of an unknown label output.

2. The method of claim 1, wherein a conventional analysis of the PPG signal may be used to:
(a) determine the critical points associated with the PPG pulse waveform and its derivatives;
(b) calculate the exhaustive set of features from the critical points, wherein the critical point associated with the PPG, VPG, and APG signals are determined using a two-moving-average method;
(c) determine the health related and interpretable engineered one dimensional (1D) features from the exhaustive set of features calculated from a difference between any two critical points in the form of amplitude, timespan, subarea, and slope features; and
(d) derive a small subset of interpretable features related to independent aspects of health and anatomy.

3. The method of claim 2, wherein the interpretable engineered one dimensional (1D) features comprise crest time, normalized crest time, SEVR, ejection duration, ejection duration index, large artery stiffness index, small artery resistance and features relating to hypertension are derived from the exhaustive set of features calculated from the x- and y-coordinates of the critical points.

4. The method of claim 2, wherein techniques for setting up covariance matrices, or principal component analysis are used to remove interpretable engineered one-dimensional (1D) features with covariance, or to select interpretable engineered one dimensional (1D) features that holds unique health or anatomy related information.

5. The method of claim 1, wherein a convolutional neural network is constructed:
(a) from a one dimensional or two-dimensional representation (via frequency domain methods, e.g. Fourier spectrum) of the PPG signal;
(b) for the extraction of feature sets from the PPG signal;
(c) for the classification of these feature sets into health state classes; and
(d) for placement of the health states in the low dimensional representative manifold.

6. The method of claim 5, wherein the input data derived from segments of the PPG signal are preprocessed and converted into a one-dimensional representation of the PPG signal, or are used to derive a two dimensional frequency domain representation of the PPG including Fourier spectrum, Lomb-Scargle periodogram or by cardiopulmonary coupling.

7. The method of claim 5, wherein a one- or two-dimensional representation of the PPG signal data is used as input data to train the CNN and to obtain the output feature set which is mapped onto the low dimensional representative framework using methods including t-SNE or PCA.

8. The method of claim 5, wherein a loss function is used to organize the manifold and to create continuity in the feature set classes, by identifying the health state regions in the low dimensional representative manifold, wherein the loss function includes triplet loss, mean square error loss, or cross entropy loss.

9. The method of claim 1, wherein a user's CNN feature set corresponding to an unknown region in the low dimensional representative manifold is compared to the interpretable engineered one dimensional (1D) features containing unique health and anatomy information as derived through the conventional analysis of the PPG signal, to evaluate the health state of the user.

10. The method of claim 9, wherein the unknown regions in the low dimensional representative manifold are assigned based on the interpretable engineered one dimensional (1D) features containing unique health and anatomy information as derived through the conventional analysis of the PPG signal corresponding to those unknown regions.

11. The method of claim 1, wherein feedback is given to users and interested third parties using displays including desktop computer display, laptop display, smartphone display, wearable device display, phone calls, text messages, emails, or web-based dashboards.

12. The method of claim 1, wherein the computational aspects of the invention are performed remotely on devices such as, but not limited to, smart wearable device, smartphone, desktop computer, laptop, or may be performed using cloud computing infrastructure.

15. A method for identifying a health status of a user using an IoT setup of interconnected electronic devices and sensors, the method comprising:
(a) collecting a user's PPG signal using a wearable device;
(b) preprocessing the PPG signal for a conventional analysis of the PPG signal to:
(i) extract at least one interpretable engineered one dimensional (1D) feature from the PPG signal;
(c) using an anomaly detection system on the PPG signal to produce a feature vector in a latent space and to produce a classification of the PPG signal corresponding to a healthy state or a state associated with another condition;
(d) constructing a low dimensional representation of the feature vector;
(e) labeling the low dimensional representation of the PPG signal corresponding to the state classified by the anomaly detection system;
(f) associating the interpretable engineered 1D features with the classification in the low dimensional representation space;
(g) resolving anomalies labeled as unknown as belonging to a healthy state or a associated with another condition; and
(h) providing feedback to the user, or to a third party, regarding a health state of the user.

16. The method of claim 15, wherein the wearable device comprises one of a fingertip pulse oximeter, an earlobe pulse oximeter, a wrist worn wearable device, or a smart ring.

17. The method of claim 15, wherein interpretable engineered 1D features comprise crest time, SEVR, ejection duration index, large artery stiffness index, small artery resistance and features relating to hypertension.

18. The method of claim 15, wherein the anomaly detection system comprises a convolutional neural network trained on PPG signal data from other users.

19. The method of claim 15, wherein constructing a low dimension representation comprises applying PCA or t-SNE to the feature vector.

20. The method of claim 1, wherein the wearable device comprises a fingertip pulse oximeter, an earlobe pulse oximeter, a wrist worn wearable device, or a smart ring.
